(19) United States
(12) Patent Application Publication
(10) Pub. No.: US 2017/0328876 A1
(43) Pub. Date: Nov. 16, 2017

(54) GAS CONCENTRATION DETECTION DEVICE AND DETECTION METHOD THEREOF

(71) Applicant: RADIANT INNOVATION INC., HSINCHU CITY (TW)

(72) Inventors: YU-CHIEN HUANG, HSINCHU CITY (TW); CHIEN-CHANG LIAO, HSINCHU CITY (TW); YU-TAI SUNG, HSINCHU CITY (TW)

(21) Appl. No.: 15/153,408

(22) Filed: May 12, 2016

(51) Int. Cl. G01N 33/00; G01N 27/04; G01N 21/61

(52) U.S. Cl. CPC G01N 33/0006; G01N 33/0073; G01N 21/61; G01N 27/04; G01N 33/0036

(57) ABSTRACT

The instant disclosure provides a gas concentration detection device including a plurality of gas concentration measurement modules and a control module. The control module coupled with the plurality of gas concentration measurement modules. Each gas concentration measurement module including: a gas chamber, a signal generating unit and a sensing unit. The control module providing a plurality of clock signals, wherein each clock signal controls the corresponding signal generating unit to correspondingly generate the medium to enter the gas chamber and pass through the gas. The sensing unit outputs a sensing signal by receiving the medium, and the control module corrects the sensing signals from the sensing units to respectively obtain corrected sensing signals, and the corrected sensing signals are integrated by the control module to obtain a gas concentration signal.

### BACKGROUND OF THE INVENTION

### 1. Field of the Invention

The present invention relates to a gas concentration detection device and detection method thereof; in particular, to a control module which selectively provides clock signals and corrects sensing units of the gas concentration detection device and detection method thereof

### 2. Description of Related Art

Currently, the principle of gas concentration detection device is to provide a light pass through gas (i.e., carbon dioxide, carbon monoxide, ammonia, methane), and the light will be decreased. Hence, the sensor could determine the variation of gas concentration according to the receiving light.

However, the sensor may senses the incorrect measuring signal because of the light source degradation, sensor consumption or other situations of the gas concentration detection device. In order to provide a stable measuring signal, the gas concentration detection device will be set up a period for correcting.

Therefore, it cannot provide measuring signal in the period for correcting of the gas concentration detection device. So that, fragmental gas concentration signals will be outputted by conventional gas concentration detection device according to the integrating measuring signal. It reduces efficiency to detect gas concentration of gas concentration detection device. For example, commercial gas concentration detection device only can provide a gas concentration signal for two times per second.

### SUMMARY OF THE INVENTION

The object of the instant invention is to solve the problems of the related art described above.

An exemplary embodiment of the present disclosure provides a gas concentration detection device comprising: a plurality of gas concentration measurement modules and a control module. The control module coupled with the plurality of gas concentration measurement modules. Each gas concentration measurement module including: a gas chamber, a signal generating unit and a sensing unit. The gas preparing for testing is introduced into a gas chamber. The signal generating unit coupled with the gas chamber for generating a medium to enter the gas chamber and pass through the gas. The sensing unit coupled with the gas chamber for receiving the medium. The control module coupled with the plurality of gas concentration measurement modules and providing a plurality of clock signals, wherein each clock signal controls the corresponding signal generating unit to correspondingly generate the medium to enter the gas chamber and pass through the gas. The sensing unit outputs a sensing signal by receiving the medium, and the control module corrects the sensing signals from the sensing units to respectively obtain corrected sensing signals, and the corrected sensing signals are integrated by the control module to obtain a gas concentration signal.

Another exemplary embodiment of the present disclosure provides a detection method of a gas concentration detection device. The gas concentration detection device comprising a plurality of gas concentration measurement modules and a control module. Each gas concentration measurement module including a gas chamber providing a gas to prepare for testing, a signal generating unit and a sensing unit. The detection method comprising the following steps: Step A: the control module providing a plurality of clock signals, wherein each clock signal controls the corresponding signal generating unit to correspondingly generate a medium to enter the gas chamber and pass through the gas. Step B: the sensing unit receiving the medium and outputting a sensing signal. Step C: the control module correcting the sensing signals from the sensing units to respectively obtain corrected sensing signals. Step D: the corrected sensing signals being integrated by the control module to obtain a gas concentration signal.

In sum, the advantages of the instant disclosure provides a gas concentration detection device and detection method thereof, which rapidly provides a continuous gas concentration signals by the control module for controlling a plurality of gas concentration measurement modules in sequence. In fact, the outputs ratio of the gas concentration signal could be 100 per second by use of the detection method in the instant disclosure.

In order to further understand the techniques, means and effects of the instant disclosure, the following detailed descriptions and appended drawings are hereby referred to, such that, and through which, the purposes, features and aspects of the instant disclosure can be thoroughly and concretely appreciated; however, the appended drawings are merely provided for reference and illustration, without any intention to be used for limiting the instant disclosure.

### BRIEF DESCRIPTION OF THE DRAWINGS

FIG. 1 shows a schematic view of a gas concentration detection device according to a first embodiment of the instant disclosure;

FIG. 2 shows a configuration view of a gas concentration detection device according to a first embodiment of the instant disclosure;

FIG. 3 shows a configuration view of a gas concentration detection device according to a second embodiment of the instant disclosure;

FIG. 4 shows a configuration view of a gas concentration detection device according to a third embodiment of the instant disclosure;

FIG. 5 shows a configuration view of a gas concentration detection device according to a fourth embodiment of the instant disclosure;

FIG. 6 shows a configuration view of a gas concentration detection device according to a fifth embodiment of the instant disclosure;

FIG. 7 shows a configuration view of a gas concentration detection device according to a sixth embodiment of the instant disclosure;

FIG. 8 shows an operating flow chart of a gas concentration detection method of any embodiment in the instant disclosure; and

FIG. 9 shows a signal correction chart of a sensing signal of any embodiment in the instant disclosure.

### DETAILED DESCRIPTION OF THE PREFERRED EMBODIMENTS

Embodiments disclosed in the instant disclosure are illustrated via specific examples as follows, and people familiar in the art may easily understand the advantages and efficacies of the instant disclosure by disclosure of the specification. The instant disclosure may be implemented or applied by other different specific examples, and each of the details in the specification may be applied based on different views and may be modified and changed under the existence of the spirit of the instant disclosure. The figures in the instant disclosure are only for brief description, but they are not depicted according to actual size and do not reflect the actual size of the relevant structure. The following embodiments further illustrate related technologies of the instant disclosure in detail, but the scope of the instant disclosure is not limited herein.

Like reference numerals refer to like elements throughout. In the drawings, the dimensions and size of each structure are exaggerated, omitted, or schematically illustrated for convenience in description and clarity. It will be understood that although the terms of first, second, and three are used herein to describe various elements or signals, but these elements or signals should not be limited by these terms. Terms are only used to distinguish one component from other components, or one signal from other signals. Therefore, a component referred to as a first component in one embodiment can be referred to as a second component in another embodiment. The terms of a singular form may include plural forms unless referred to the contrary. In addition, the meaning of ‘comprise’, ‘include’, or ‘have’ specifies a property, a region, a fixed number, a step, a process, an element and/or a component but does not exclude other properties, regions, fixed numbers, steps, processes, elements and/or components

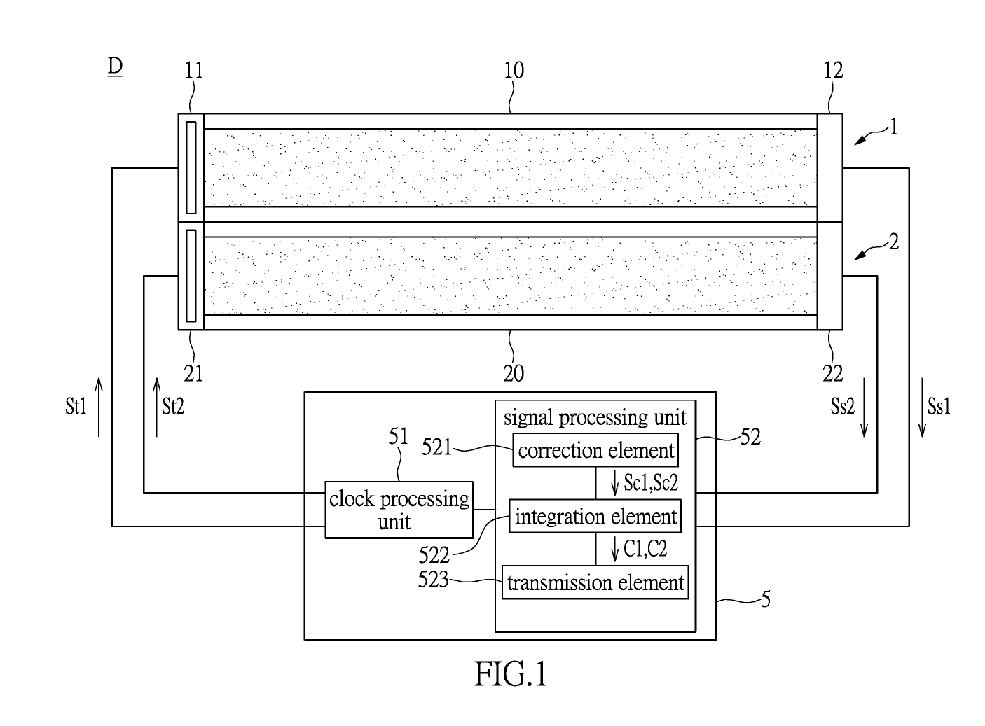

First, please refer to FIG. 1. FIG. 1 shows a schematic view of a gas concentration detection device according to a first embodiment of the instant disclosure. The first embodiment provides a gas concentration detection device D for continuously detecting and outputting a gas concentration signal. The gas concentration detection device D including a plurality of gas concentration measurement modules and a control module 5,and the control module 5 coupled with the plurality of gas concentration measurement modules. In addition, it should be appreciated that the gas concentration detection device D of the instant disclosure can be used in medical industry for monitoring respiratory gases, or used in industrial industry for measuring industrial waste gases. The application field for the gas concentration detection device of this instant disclosure is not limited.

To be specific, the gas concentration detection device D including a plurality of gas concentration measurement modules, and each gas concentration measurement module has the same structure, so that one of the gas concentration measurement modules will be introduced as below (for example, a first gas concentration measurement module 1). In addition, each gas concentration measurement module is introduced a gas from the same source or has similar property. The first gas concentration measurement module 1 includes a first gas chamber 10, a first signal generating unit 11 and a first sensing unit 12. The first gas chamber 10 respectively coupled with the first signal generating unit 11 and the first sensing unit 12. In this embodiment, the first signal generating unit 11 and the first sensing unit 12 are disposed on the first gas chamber 10. More precisely, the first signal generating unit 11 and the first sensing unit 12 are disposed on the opposite side of the first gas chamber 10. Then a gas preparing for testing is introduced into the first gas chamber 10. In other embodiments, each gas concentration measurement module can have different structures. It could be adjusted to the structure of gas concentration measurement module by those of ordinary skill in the art. However, the instant disclosure is not limited thereto. It is worthwhile to mention that the first signal generating unit 11 also could be disposed outside of the first gas chamber 10, as long as the first signal generating unit 11 could generate medium to enter the gas chamber. And the first sensing unit 12 also could be disposed outside of the first gas chamber 10, as long as the first sensing unit 12 could receive the medium pass through gas.

The first signal generating unit 11 generates the medium to enter the first gas chamber 10 and pass through the gas to be tested. And the first sensing unit 12 receives the medium which pass through the gas and provides a sensing signal according to the medium sensed by a variation of gas concentration.

For example, the gas concentration measurement module is Non-Dispersive Infrared (“NDIR”) technique, which is a method of calculating gas concentration according to measure the variation of infrared which pass through the gas to be tested. Therefore, the first signal generating unit 11 is a light emitting element (i.e., infrared light-emitting element), the first sensing unit 12 is a light sensing element. The light emitting element emits a light to a gas and passes through the gas to enter the light sensing element. The light sensing element outputs a light sensing signal to the control module 5 according to the receiving light.

For another example, the gas concentration measurement module detecting the gas concentration by a resistor element, which calculating the gas concentration signals by resistance value variation. To be specific, the variation of resistance value is based on absorption between the resistance surface and the gas under high temperature. Therefore, the first signal generating unit 11 is a heating element, the first sensing unit 12 is a resistor. When the heating element generates a thermal energy to the resistor and pass through a gas, thereby the resistance value will be changed by the gas absorb on the resistance surface or break out the resistance surface. Next, the resistor generates an electric signal according to the changed current value, and the control module calculates the gas concentration according to determine the value of the electric signal.

It is worthwhile to mention that in this embodiment, the plurality of gas concentration measurement modules of the gas concentration detection device D use the same technique to detect the gas concentration. However, the instant disclosure is not limited thereto. In other embodiments, the plurality of gas concentration measurement modules of the gas concentration detection device D can use different techniques to detect the gas concentration. For example, the first gas concentration measurement module 1 detects gas concentration by NDIR technique, and the second gas concentration measurement module 2 detects gas concentration by a resistor. Thus, the first gas concentration measurement module 1 has a different design structure than the second gas concentration measurement module 2.

The control module 5 (i.e., a controller or a microcontroller) provides the plurality of clock signals to selectively control the plurality of signal generating units. To be specific, the control module 5 includes a clock processing unit 51 and a signal processing unit 52, the clock processing unit 51 respectively provides different clock signals to the signal generating unit by the changing time. Each clock signal controls the corresponding signal generating unit to correspondingly generate the medium to enter the gas chamber and pass through the gas. And the signal processing unit 52 receives the sensing signals from the sensing units and processes the sensing signals to provide a gas concentration signal.

Please refer to FIG. 1. The clock processing unit 51 of the control module 5 respectively provides different clock signals to the signal generating units, and each signal generating unit respectively provides the medium enter to the gas chambers pass through the gas in sequence. The sensing unit respectively outputs sensing signals to the control module 5 by respectively receives the mediums.

The signal processing unit 52 of the control module 5 receives the sensing signals from the different sensing units. Then, the signal processing unit 52 respectively corrects each sensing signal from the sensing unit to respectively obtain corrected sensing signal.

Furthermore, the signal processing unit 52 further includes a correction element 521, an integration element 522 and a transmission element 523. The integration element 522 coupled with the correction element 521 and the transmission element 523. The correction element 521 respectively corrects the sensing signals and respectively provides a plurality of corrected sensing signals. The process of the correction element 521 calculating the sensing signals to obtain the corrected sensing signals will be described as below. The integration element 522 receives and integrates the corrected sensing signals to obtain a gas concentration signal. And the transmission element 523 outputs the gas concentration signal.

It is worthwhile to mention that the signal generating units simultaneously generate the mediums to the gases according to the clock signals generated by the clock processing unit 51 provides to the signal generating units. Or the signal generating units simultaneously do not generate the mediums to the gases. Either, the signal generating units alternatively generate the mediums to the gases. For example, when one signal generating unit generates medium, another signal generating unit does not generate medium. It could be adjusted to selectively provides medium from different signal generating units according to the clock signals by those of ordinary skill in the art.

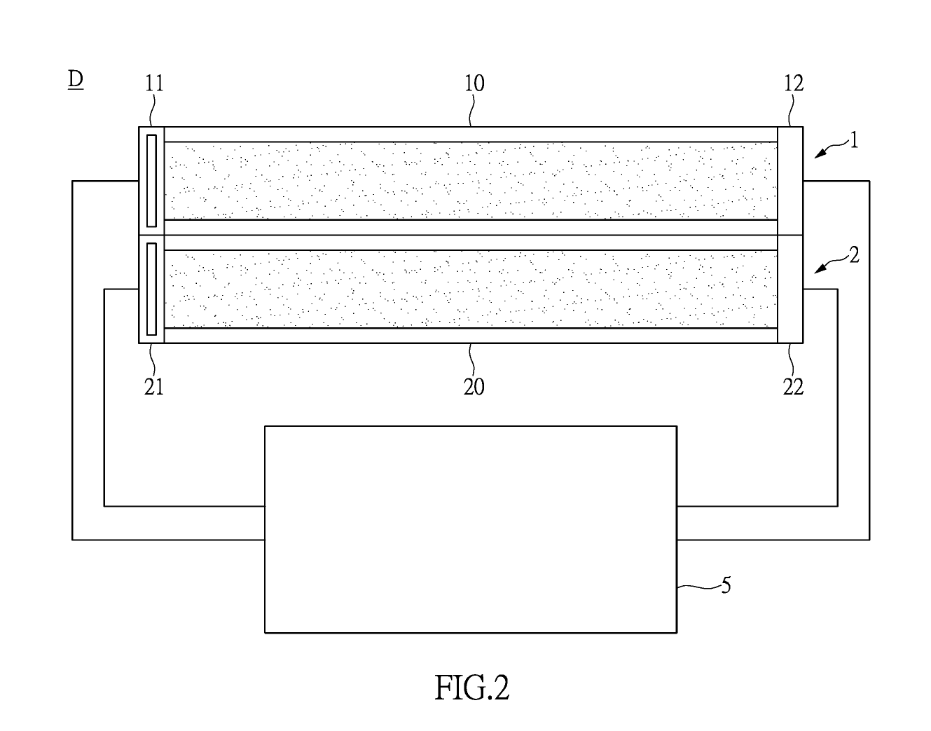

Please refer to FIG. 2. FIG. 2 shows a configuration view of a gas concentration detection device according to a first embodiment of the instant disclosure. In this embodiment, two of the plurality of the signal generating units of the plurality of gas concentration measurement modules disposed on the same lateral side of the gas concentration detection device. For example, the first embodiment of the gas concentration detection device (as shown in FIG. 2) includes the first gas concentration measurement module 1 and the second gas concentration measurement module 2. The second gas concentration measurement module 2 includes a second gas chamber 20, a second signal generating unit 21 and a second sensing unit 22. The second gas concentration measurement module 2 has almost same structure as the first gas concentration measurement module 1, it will not be described in detail herein. To be specific, in the first embodiment of the instant disclosure. The first gas concentration measurement module 1 assembled with the second gas concentration measurement module 2 and the first signal generating unit 11 and a second signal generating unit 21 disposed on the same lateral side of the gas concentration detection device, so that the first sensing unit 12 and second sensing unit 22 disposed on the another same lateral side of thereon.

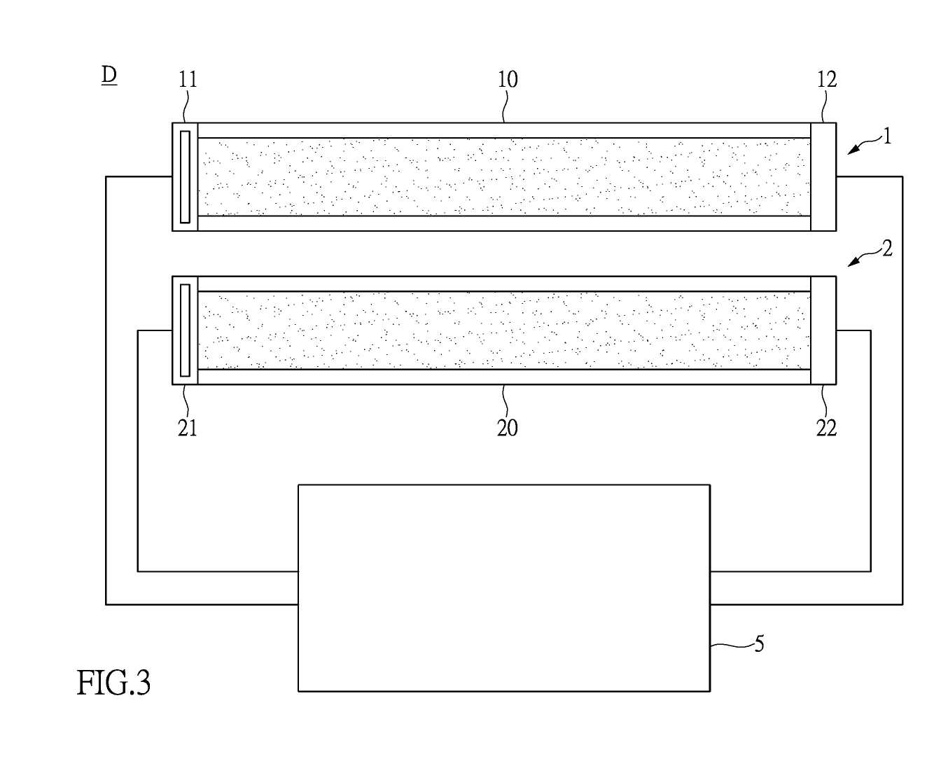

Please refer to FIG. 3. FIG. 3 shows a configuration view of a gas concentration detection device according to a second embodiment of the instant disclosure. Plurality of gas concentration measurement modules of the second embodiment are different with the first embodiment shown in FIG. 2, which is the first gas concentration measurement module 1 and the second gas concentration measurement module 2 set apart from each other.

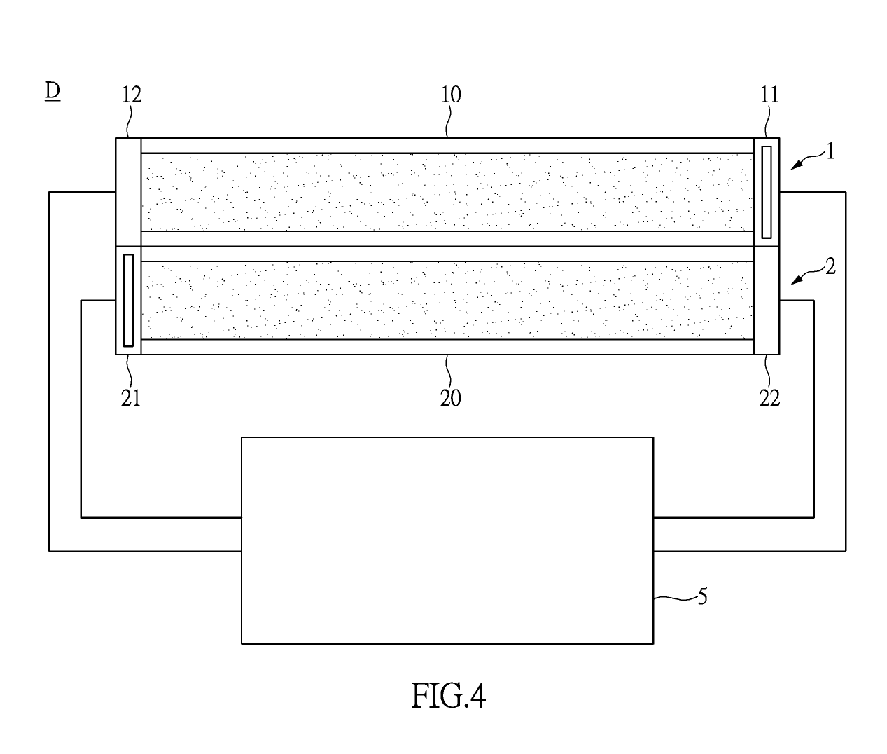

Please refer to FIG. 4. FIG. 4 shows a configuration view of a gas concentration detection device according to a third embodiment of the instant disclosure. In the third embodiment of the instant disclosure, two of the plurality of signal generating units of the plurality of gas concentration measurement modules disposed diagonally. For example, the first signal generating unit 11 and a second signal generating unit 21 disposed on a diagonal line of the gas concentration detection device and the first sensing unit 12 and second sensing unit 22 disposed on the another diagonal line thereon, thereby these two diagonal lines are arranged as a cross-diagonal matrix.

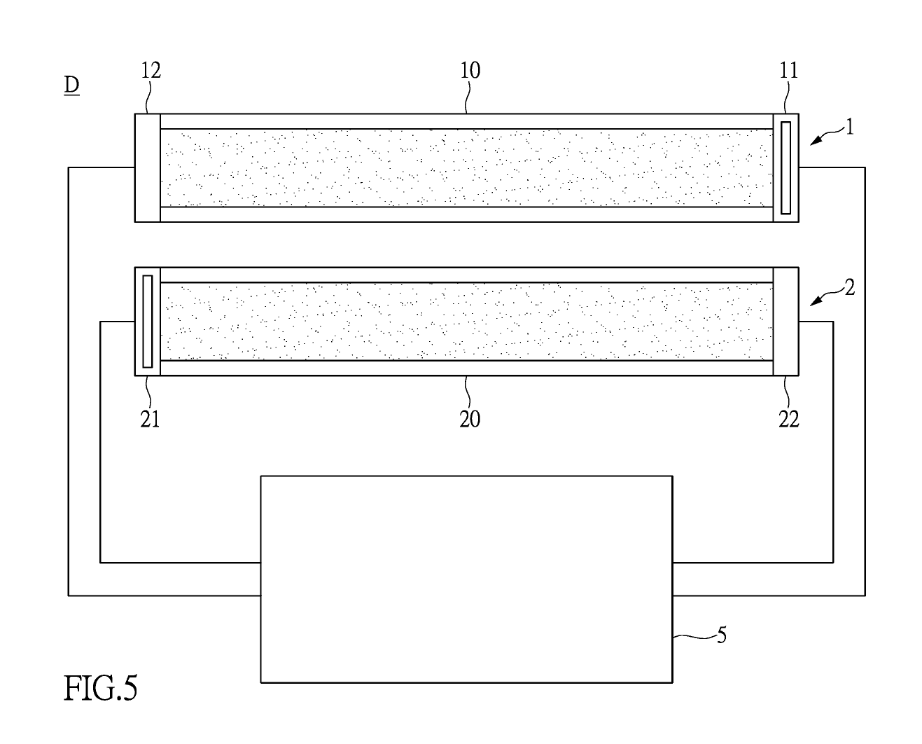

Please refer to FIG. 5. FIG. 5 shows a configuration view of a gas concentration detection device according to a fourth embodiment of the instant disclosure. Plurality of gas concentration measurement modules of the fourth embodiment are different with the third embodiment shown in FIG. 4, which is the first gas concentration measurement module 1 and the second gas concentration measurement module 2 set apart from each other.

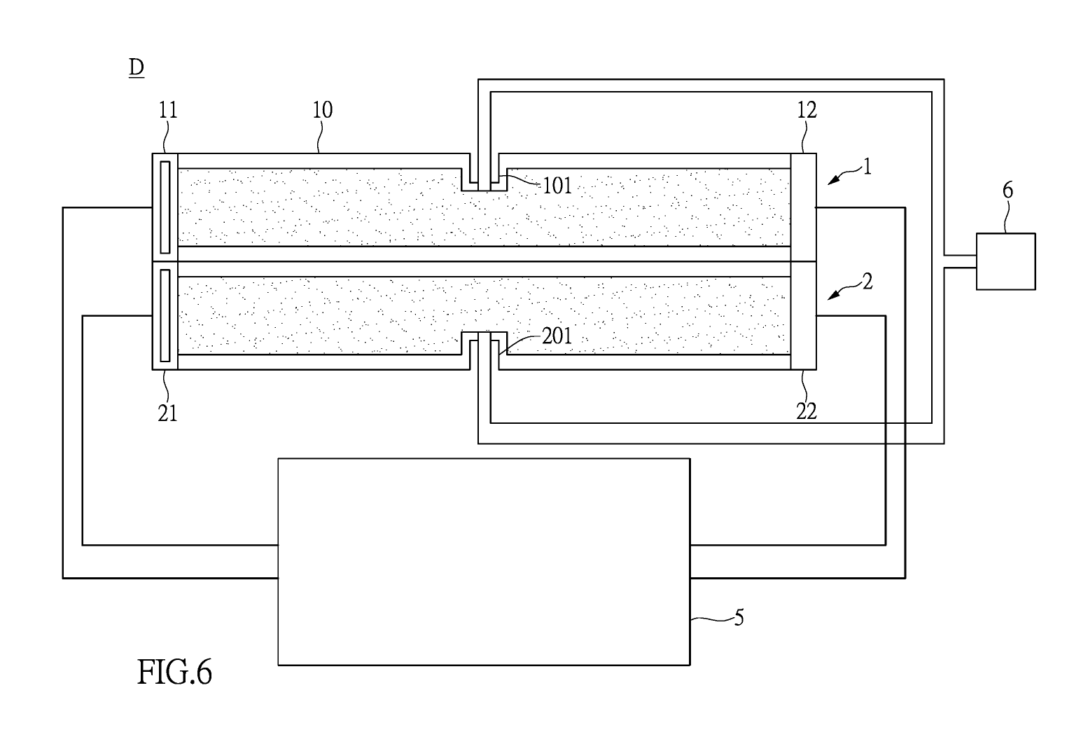

Please refer to FIG. 6. FIG. 6 shows a configuration view of a gas concentration detection device according to a fifth embodiment of the instant disclosure. The gas concentration detection device of the fifth embodiment is different with the first embodiment shown in FIG. 2, which includes a pressure module 6 (i.e., a pump apparatus or a fan apparatus). The pressure module 6 coupled with the plurality of gas concentration measurement modules. The gas chamber of each gas concentration measurement module has at least one air vent thereon. Then, the pressure module 6 could introduce the gas pass through the at least one air vent of the plurality of gas chambers to be a sample that preparing for testing.

To be specific, as shown in FIG. 6. In the fifth embodiment, the first gas concentration measurement module 1 assembled with the second gas concentration measurement module 2. Hence, the pressure module 6 respectively introduces the gases by the first air vent 101 of the first gas concentration measurement module 1 and the second air vent 201 of the second gas concentration measurement module 2. Or, the pressure module 6 respectively withdraws the gas from the gas chambers by the first air vent 101 of the first gas concentration measurement module 1 and the second air vent 201 of the second gas concentration measurement module 2. The first air vent 101 disposed on a wall of the first gas chamber 10, and the wall far away from the second gas concentration measurement module 2. The second air vent 201 disposed on a wall of the second gas chamber 20, and the wall far away from the first gas concentration measurement module 1.

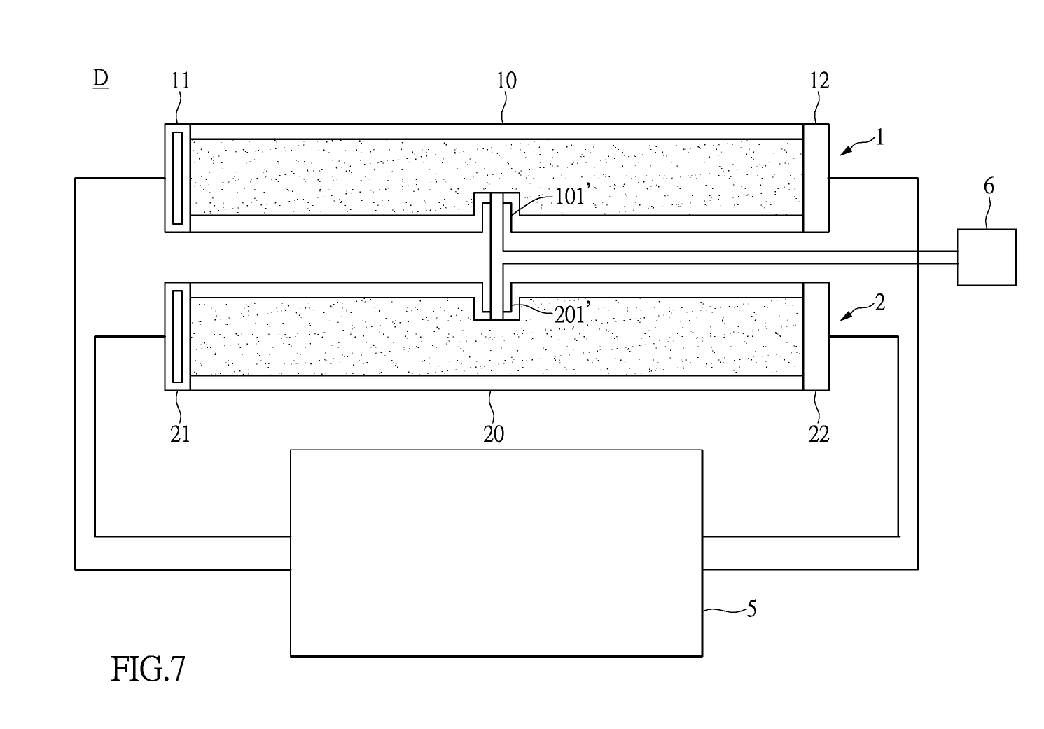

Please refer to FIG. 7. FIG. 7 shows a configuration view of a gas concentration detection device according to a sixth embodiment of the instant disclosure. The gas concentration detection device of the sixth embodiment is different with the fifth embodiment shown in FIG. 6, which is the gas concentration measurement module 1 and the second gas concentration measurement module 2 set apart from each other. Moreover, the first air vent 101′ disposed on a wall adjacent to the second gas concentration measurement module 2 of the first gas chamber 10′, and the second air vent 201′ disposed on a wall adjacent to the first gas concentration measurement module 1 of the second gas chamber 20′.

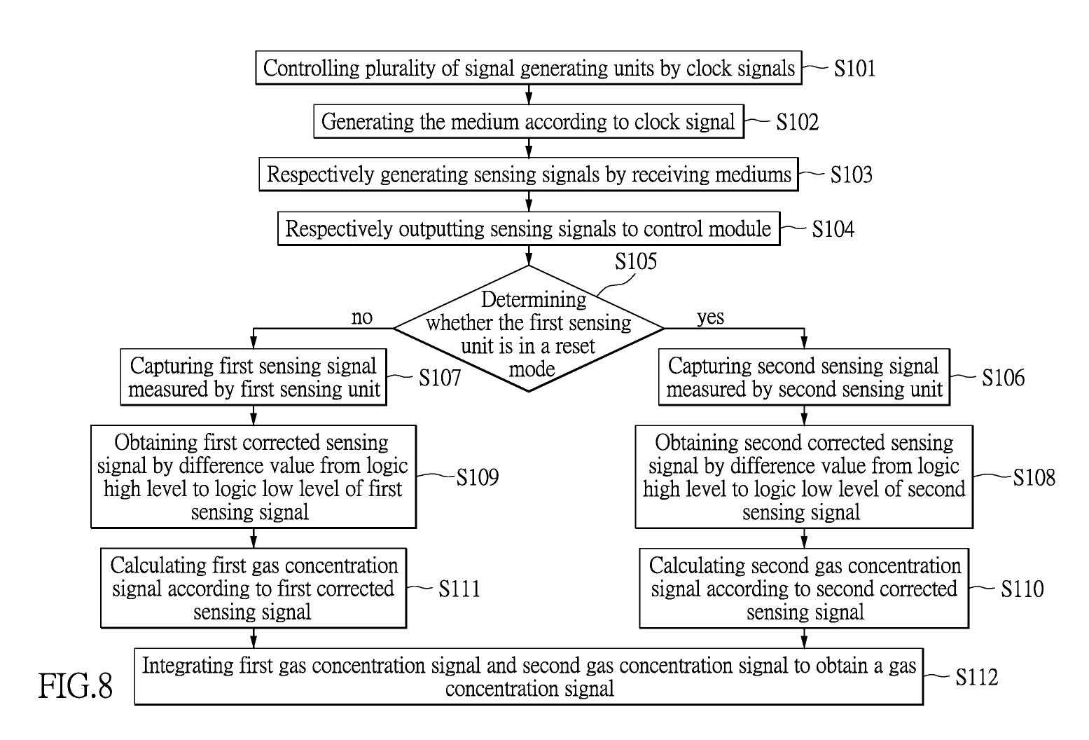

The process of a gas concentration detection device D of the detection method will be described in details as below. As shown in FIG. 1. Please refer to FIG. 8. FIG. 8 shows an operating flow chart of a gas concentration detection method of any embodiment in the instant disclosure. In the embodiments of the instant disclosure, the gas preparing for testing already pre-introduced into the gas chamber by the pressure module. In step S101, the control module provides the plurality of clock signals, and the clock signals selectively controls the plurality of signal generating units. Specifically, the clock processing unit 51 of the control module 5 provides a first clock signal St1 to the first signal generating unit 11, and also provides a second clock signal St2 to the second signal generating unit 21. It is worthwhile to mention that the first clock signal St1 and the second clock signal St2 are the same signals, quite different signals or even alternates same signals by changing time. It could be adjusted to the signals by those of ordinary skill in the art. In this embodiment, the first clock signal St1 and the second clock signal St2 are different signals, so that the first signal generating unit 11 and the second signal generating unit 21 alternately provides the mediums.

In step S102, when the first signal generating unit 11 and the second signal generating unit 21 respectively receives different clock signals St1, St2, and the mediums are respectively generated to the gas preparing for testing according to the different clock signals. Next, goes to the step S103.

In step S103, the sensing unit respectively generated the sensing signals according to the receiving mediums. As shown in FIG. 1, the first signal generating unit 11 generates the medium pass through the gas of the first gas chamber 10 according to the first clock signal St1, and the first sensing unit 12 generates a first sensing signal Ss1 by the receiving medium. At the same time, the second signal generating unit 12 generates the another medium pass through the gas of the second gas chamber 20 according to the second clock signal St2, and the second sensing unit 22 generates a second sensing signal Ss2 by the receiving medium.

In step S104, the sensing unit respectively outputs sensing signals to the control module 5. Specifically, the first sensing unit 12 outputs the first sensing signal Ss1 to the signal processing unit 52 of the control module 5. At the same time, the second sensing unit 22 outputs the second sensing signal Ss2 to the signal processing unit 52 of the control module 5.

In step S105, the control module 5 receives the first sensing signal Ss1 and the second sensing signal Ss2 for determining whether the first sensing unit 12 is in a reset mode. To be specific, the first sensing unit 12 can be reset by automatic reset mode, so that the first sensing unit 12 will automatically reset itself in a period. Or the first sensing unit 12 receives a reset signal by central controller to enter the reset mode (not shown in FIG. 1). Hence, a logic high level of the first sensing signal Ss1 is outputted in the work mode of the first sensing unit 12, and a logic low level of the first sensing signal Ss1 is outputted in reset mode of the first sensing unit 12. It is worthwhile to mention that the second sensing unit 22 could switch the work mode and the reset mode by using the same. It will not be described in detail herein.

On the other hand, the clock processing unit 51 can control the first signal generating unit 11 do not generate the medium in reset mode of the first sensing unit 12, thereby it saving power consumed for the gas concentration detection device D.

A correction element 521 determines the first sensing unit 12 in the work mode or the reset mode according to the first sensing signal Ss1, and determines the second sensing unit 22 in the work mode or the reset mode according to the second sensing signal Ss2.

If the correction element 521 determines the first sensing unit 12 is in the reset mode, then goes to the S106; otherwise, if the correction element 521 determines the first sensing unit 12 is not in the reset mode, goes to the step S107.

In step S106, if the first sensing unit 12 is in the reset mode, the control module 5 correspondingly captures the second sensing signal Ss2 measured by the second sensing unit 22. Specifically, when the first sensing unit 12 is in the reset mode so that the second sensing unit 22 is in the work mode. Therefore, the correction element 521 of the control module 5 captures the second sensing signals Ss2. Next, goes to the step S108.

In step S108, the corrected sensing signals obtained by the control module 5 according to the difference value from the logic high level to the logic low level of the second sensing signal Ss2. Specifically, the correction element 521 determines the logic high level and the logic low level of the second sensing signal Ss2 and calculates the difference between both to obtain the second corrected sensing signal Sc2. Next, goes to the step S110. In step S110, the second gas concentration signal C2 calculated by the integration element 522 of the control module 5 according to the second corrected sensing signal Sc2, and goes to the step S112.

In step S107, if the first sensing unit 12 is not in the reset mode, the control module 5 correspondingly captures the first sensing signal Ss1 measured by the first sensing unit 12. Specifically, when the first sensing unit 12 is not in the reset mode so that the first sensing unit 12 is in the work mode. Therefore, the correction element 521 of the control module 5 captures the first sensing signals Ss1. Next, goes to the step S109.

In step S109, the corrected sensing signals obtained by the control module 5 according to the difference value from the logic high level to the logic low level of the first sensing signal Ss1. Specifically, the correction element 521 determines the logic high level and the logic low level of the first sensing signal Ss1 and calculates the difference between both to obtain the first corrected sensing signal Sc1. Next, goes to the step S111. In step S111, the first gas concentration signal C1 calculated by the integration element 522 of the control module 5 according to the first corrected sensing signal Sc1, and goes to the step S112.

In step S112, the first gas concentration signal C1 and the second gas concentration signal C2 received by the transmission element 523 of the control module 5. Then, a gas concentration signal Ct obtained by the transmission element 523 for integrating the first gas concentration signal C1 and the second gas concentration signal C2.

It is worthwhile to mention that the control module 5 also does not include integration element 522 and transmission element 523. Thus, in the step S107 and S108, the correction element 521 directly captures the first sensing signal Ss1 and the second sensing signal Ss2 and obtains the gas concentration signal by terminal processing unit from outputted first corrected sensing signal Sc1 and second corrected sensing signal Sc2.

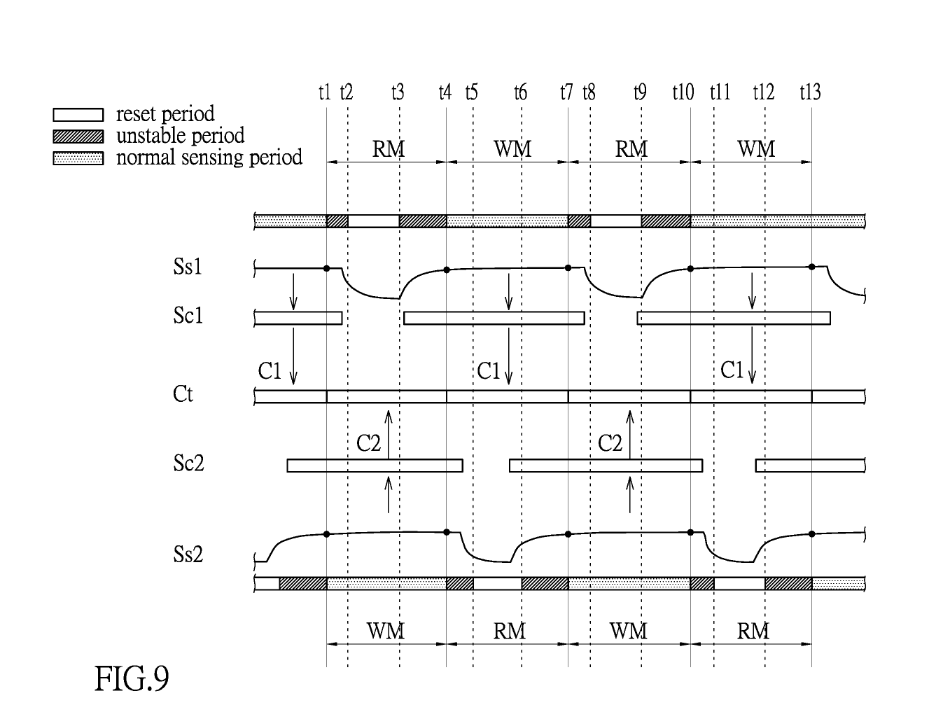

The process of the control module 5 receives different sensing signals and obtains the gas concentration signal Ct will be described as below. As shown in FIG. 1 and FIG. 8. Please refer to FIG. 9. FIG. 9 shows a signal correction chart of a sensing signal of an embodiment in the instant disclosure.

The signal generating unit (i.e., the first signal generating unit 11 and the second signal generating unit 21 described above) respectively generates different mediums according to the clock signals, and the sensing unit (i.e., the first sensing unit 12 and the second sensing unit 22 described above) respectively receives different mediums for selectively into the reset mode RM or work mode WM in sequence.

To be specific, the reset mode RM has an unstable period and a reset period. In the reset period, the sensing unit is in reset state and reset an internal parameter thereof. At this time, the sensing unit cannot correctly sense the medium and output a sensing signal after receives the medium. Furthermore, the unstable period between completely reset the sensing unit to enter the work mode, or between the work mode and the reset mode of the sensing unit. In the unstable period, the sensing unit also cannot correctly sense the medium and output a sensing signal. For example, the first sensing signal Ss1 outputted by the first sensing unit 11 might provide an unstable signal for rapidly increasing or rapidly decreasing in the unstable period. Otherwise, the work mode WM is a normal sensing period, and the sensing signal outputted by the sensing unit is relatively stable.

Please refer to FIG. 1 and FIG. 9. The first sensing unit 12 and the second sensing unit 22 are respectively into different mode by changing time. At time point t1 to time point t4, the first sensing unit 12 is in the reset mode RM, so that the first sensing unit 12 cannot correctly provide the first sensing signal Ss1. Specifically, the first sensing unit 12 is in the unstable period at time point t1 to time point t2, and the first sensing unit 12 is in the reset period at time point t2 to time point t3, and the first sensing unit 12 is also in the unstable period at time point t3 to time point t4. At this time, the second sensing unit 22 is in the work mode WM and provides the second sensing signal Ss2 according to the receiving medium. The first sensing unit 12 switches to the work mode WM at time point t4 to time point t7. On the other hand, the second sensing unit 22 is into the reset mode RM. Specifically, the second sensing unit 22 is in the unstable period at time point t4 to time point t5, and the second sensing unit 22 is in the reset period at time point t5 to time point t6, and the second sensing unit 22 is also in the unstable period at time point t6 to time point t7.

At time point t7 to time point t10, the first sensing unit 12 is into reset mode RM again and resets the internal parameters thereof. Then, the second sensing unit 22 switches to the work mode WM and senses the gas concentration according to the receiving medium. At time point t10 to time point t13, the first sensing unit 12 is into work mode WM again and the second sensing unit 22 is into reset mode RM. And so on, the first sensing unit 12 is alternatively under reset mode RM or work mode WM in sequence, and simultaneously the second sensing unit 22 is correspondingly under reset mode RM or work mode WM. As long as one sensing units is in the reset mode RM, and another sensing units can normally sense the gas concentration in the work mode WM to complete invention.

Next, the signal processing unit 52 receives the first sensing signal Ss1 outputted by the first signal generating unit 11 and calculates the logic high level and the logic low level of the first sensing signal Ss1. And the first corrected sensing signal Sc1 calculated by the signal processing unit 52 according to the difference value from the logic high level to the logic low level. In addition, the process of calculating the second corrected sensing signal Sc2 as same as the first corrected sensing signal Sc1, it will not be described in detail herein.

The signal processing unit 52 captures different corrected sensing signals in sequence, and integrates the corrected sensing signals to obtain a gas concentration signal. To be specific, the signal processing unit 52 determines the first sensing signal 12 is in the reset mode RM at the time point t1 to time point t4, therefore the signal processing unit 52 captures the second corrected sensing signal Sc2 for calculating the second gas concentration signal C2. Then, the signal processing unit 52 determines the second sensing signal 22 is in the reset mode RM, therefore the signal processing unit 52 captures the first corrected sensing signal Sc1 for calculating the first gas concentration signal C1. And the gas concentration signal Ct obtained by the signal processing unit 52 for integrating the first gas concentration signal C1 and the second gas concentration signal C2

There is illustrated a typical embodiment of gas concentration detection device D above described by those skilled in the art, and the embodiments have various changes for subsequent application based on the invention.

In addition, the gas concentration detection device of other embodiments in instant invention can include three or more gas concentration measurement modules. While one gas concentration measurement module is in reset mode and another gas concentration measurement module in is work mode, it will be completely the invention. It could be adjusted to the numbers of gas concentration measurement module by those of ordinary skill in the art, and it is not limited herein.

In sum, the advantages of the instant disclosure provides a gas concentration detection device and detection method thereof, which rapidly provides a continuous gas concentration signals by the control module for controlling a plurality of gas concentration measurement modules in sequence. In fact, the outputs ratio of the gas concentration signal could be 100 per second by use of the detection method in the instant disclosure.

Another advantage of the instant disclosure is that, the sensing unit of the gas concentration detection device can regularly into reset mode for self-correction. Therefore, the inside temperature of the sensing unit will keep to safe range for extending the usage life of the element. Besides, the signal generating unit cannot provide medium when correspondingly sensing unit is in the reset mode, so that saving the electrical consumption for the gas concentration detection device.

The descriptions illustrated supra set forth simply the preferred embodiments of the present invention; however, the characteristics of the present invention are by no means restricted thereto. All changes, alternations, or modifications conveniently considered by those skilled in the art are deemed to be encompassed within the scope of the present invention delineated by the following claims.

What is claimed is:

1. A gas concentration detection device, comprising:
a plurality of gas concentration measurement modules, each gas concentration measurement module including:
a gas chamber, wherein a gas preparing for testing is introduced into a gas chamber;
a signal generating unit coupled with the gas chamber for generating a medium to enter the gas chamber and pass through the gas; and
a sensing unit coupled with the gas chamber for receiving the medium; and
a control module coupled with the plurality of gas concentration measurement modules and providing a plurality of clock signals, wherein each clock signal controls the corresponding signal generating unit to correspondingly generate the medium to enter the gas chamber and pass through the gas;
wherein the sensing unit outputs a sensing signal by receiving the medium, and the control module corrects the sensing signals from the sensing units to respectively obtain corrected sensing signals, and the corrected sensing signals are integrated by the control module to obtain a gas concentration signal.

2. The gas concentration detection device according to claim 1, wherein the mediums are generated from the signal generating units respectively according to the clock signals provided by the control module.

3. The gas concentration detection device according to claim 1, wherein the sensing signal has a logic high level and a logic low level, and the corrected sensing signals obtained by the control module according to the difference value from the logic high level to the logic low level.

4. The gas concentration detection device according to claim 1, wherein two of the plurality of signal generating units of the plurality of gas concentration measurement modules disposed on the same lateral side of the gas concentration detection device.

5. The gas concentration detection device according to claim 1, wherein two of the plurality of signal generating units of the plurality of gas concentration measurement modules disposed diagonally.

6. The gas concentration detection device according to claim 1, further including:
a pressure module coupled with the plurality of gas concentration measurement modules, wherein the pressure module is disposed on at least one air vent of each gas chamber for introducing the gas.

7. The gas concentration detection device according to claim 1, wherein the signal generating unit of the gas concentration measurement module is a light emitting element for generating a light to pass through the gas, and the light sensing unit is a light sensing element for outputting a light sensing signal according to the light.

8. The gas concentration detection device according to claim 1, wherein the signal generating unit of the gas concentration measurement module is a heating element for generating a thermal energy to pass through the gas, and the sensing unit is a resistor for providing an electric signal according to resistance value of the resistor.

9. A detection method of a gas concentration detection device, the gas concentration detection device comprising a plurality of gas concentration measurement modules and a control module, each gas concentration measurement module including a gas chamber providing a gas to prepare for testing, a signal generating unit and a sensing unit, the detection method comprising the following steps:
Step A: the control module providing a plurality of clock signals, wherein each clock signal controls the corresponding signal generating unit to correspondingly generate a medium to enter the gas chamber and pass through the gas;
Step B: the sensing unit receiving the medium and outputting a sensing signal;
Step C: the control module correcting the sensing signals from the sensing units to respectively obtain corrected sensing signals; and
Step D: the corrected sensing signals being integrated by the control module to obtain a gas concentration signal.

10. The method as claimed in claim 9, wherein in the step A, the mediums are respectively generated from the signal generating units according to the clock signals provided by the control module.

11. The method as claimed in claim 9, wherein in the step C, the sensing signal has a logic high level and a logic low level, and the corrected sensing signals obtained by the control module according to the difference value from the logic high level to the logic low level.
